(19) United States
(12) Patent Application Publication
(10) Pub. No.: US 2002/0023648 A1
(43) Pub. Date: Feb. 28, 2002

(54) Gas pressure regulator incorporating flow meter

(75) Inventor: David Komesaroff, South Yarra (AU)

Correspondence Address: E. Victor Indiano, Esq., Suite 850, 1 North Pennsylvania Street, Indianapolis, IN, 46204, US

(73) Assignee: Medical Developments Australia, Pty, Ltd

(21) Appl. No.: 09/733,826

(22) Filed: Dec. 9, 2000

(30) Foreign Application Priority Data
Dec. 9, 1999 (AU) PQ4561

(51) Int. Cl. A62B 18/02; A62B 7/00; A61M 16/00; F16K 31/02

(52) U.S. Cl. 128/205.25; 128/204.21

(57) ABSTRACT

A gas supply regulator/flowmeter for supplying gases to a patient from a gas cylinder, said regulator/flowmeter incorporating means to regulate gas pressure and means to control a main rate of flow of gas at a single constant flow rate or a plurality of different flow rates. A valve means is provided hereby to provide, upon activation, and downstream of the means to control the main rate of flow of gas, a temporary burst of increased flow of gas to the patient via associated resuscitation or oxygen therapy equipment.

### TECHNICAL FIELD/BACKGROUND OF THE INVENTION

This invention relates to a pressure regulator/flowmeter incorporating a flowmeter (hereinafter referred to generally as a regulator/flowmeter), and more particularly, but not exclusively, an oxygen pressure regulator/flowmeter, and also more particularly, but not exclusively a regulator/flowmeter for use with portable oxygen resuscitators or oxygen therapy units (breathing apparatus) as may be carried by emergency vehicles, such as ambulances, or by boats such as may be used for diving purposes, or also in factories, executive offices, sporting grounds, underground mines or chambers. Such regulator/flowmeters are for the purpose of delivering oxygen from an oxygen cylinder, to patients at the scene of accidents or in other emergency situations, via an oxygen therapy mask (often fitted with an oxygen reservoir bag) OR a self-inflating bag (often fitted with an oxygen reservoir bag) commonly referred to as a bag Valve Mask system (BVM) OR to an inflatable breathing bag. The choice of the delivery apparatus is influenced by whether or not the patient is breathing and the experience of the operator. Although hereinafter the invention will be described as applied to a pressure regulator/flowmeter for oxygen supply it is also applicable to other gases such as air or mixtures of oxygen and other gases. Although hereinafter the invention will be described as applied to a pressure regulator/flowmeter for oxygen supply it is also applicable to other gases such as air or mixtures of oxygen and other gases.

Such known regulator/flowmeters incorporate adjustable valve means for metering the flow of oxygen to the patient in accordance with what the administering medical or ambulance officer decides is appropriate including the method of administering oxygen.

Such regulator/flowmeters have a plurality of settings for volumes of oxygen to be administered, for example from zero flow through a low setting of 3 litres/minute through intermediate settings of 3, 8 and 15 litres/minute and through to a maximum setting of 25 litres/minute. For systems having therapy masks (sometimes known a the Hudson type) and with an oxygen reservoir bag, the selected flow rate is usually set to litres/minute. Occasionally higher flow rates may be selected to keep the oxygen reservoir bag adequately full.

With other systems fitted with an oxygen reservoir bag, the flow rate of oxygen is usually set at litres/minute. However frequently this needs to be increased to litres/minute to keep the oxygen reservoir bag full, particularly if the rate of compressing the recoil bag is too deep or too high a rate. In many situations a setting of 8 litres/minute is sufficient, although there is a tendency if the patient is receiving insufficient oxygen to increase the flow to one of the higher settings and to thereafter unnecessarily leave the regulator/flowmeter at that higher setting then all that was required was a temporary short increased burst or flush of oxygen. The result is that the supply of oxygen is more rapidly exhausted.

In many emergency situations the regulator/flowmeter is fully opened for the purposes of providing high oxygen flows, for example 15 or 25 litres/minute. Although the 25 litres/minute is much greater than the usually-necessary rate of about 8 litres/mute, the additional flow is believed to be necessary in certain circumstances for a brief period of time, but not continuously.

Although fully opening the pressure regulator/flowmeter to deliver oxygen at a flow rate of 25 litres/minute achieves the objective for the relatively brief period of time required, a flow rate of 25 litres/minute severely reduces the amount of oxygen supply. Fortunately in many cases the premature depletion of oxygen results in nothing more harmful than increased oxygen costs caused by the increased oxygen usage. However, the premature depletion of oxygen has the potential to adversely affect the patient's health condition if oxygen depletion occurs before the patient arrives at a place where there is an additional oxygen supply. Such a situation may occur when a large amount of time is required to travel from the spot where the oxygen was first applied (e.g. the Patient's house), to a health care facility such as a hospital, that has an additional available oxygen supply.

On a particularly long trip, there is a greater likelihood that the delivery of oxygen at 25 litres/mixture will result in the oxygen being fully depleted before the patient arrives at the hospital than if the oxygen were delivered at 8 litres/minute. For example, a typical oxygen tank will theoretically be able to deliver oxygen to a patient for over 50 minutes at 8 litres/minute, but only 17.5 minutes at 25 litres/minute. Unless the emergency medical technician or his ambulance vehicle has a spare oxygen tank, this premature depletion of the oxygen tank could result in the patient being deprived of necessary oxygen for a period of time.

Another drawback of prior art oxygen delivery systems, is that it is somewhat complicated and tricky to use, as it requires a person to understand oxygen flow rates sufficiently well to be able to set the control knob at an appropriate flow rate. Although the act of setting a control knob to provide the proper oxygen flow rate does not provide much of an obstacle to a skilled emergency medical technician, such as the emergency technicians who operate ambulances, the device may be too complicate to be used easily by personnel who are not as well trained, and who do not use oxygen delivery systems frequently.

By making the device more simple to use, the applicant believes that the device will be useful not only for trained emergency medical technicians, but also for lesser trained care givers, who have only limited amounts of training, such as the limited amount of training that is typically given in a “Red Cross®”-type CPR class.

In recent years, medical science has discovered that the severity of acute and life-threatening health condition can be reduced significantly if the patient undergoing the adverse health condition is given sufficient oxygen in a timely manner. In this regard, the timely introduction of oxygen to patients suffering from heart attacks, severe congestive heart failure, and strokes can significantly reduce the likelihood of the patient dying from the condition, and can further reduce the long-term damage suffered by the patient as a result of that condition. It has further been found that the severity of the long term affects of adverse health conditions will be reduced substantially if the oxygen is given to the patient as soon as possible. As a corollary, a prolonged delay in giving oxygen increases the severity of the long-term affect because longer periods of oxygen deprivation usually result in substantial cellular and tissue death due to the oxygen depravation.

One of the primary factors that determines the amount of elapsed time between the recognition and on-set of the adverse health condition and the application of oxygen is the amount of time required for the oxygen containing emergency vehicle to drive to the place where the patient is located. It follows naturally that the availability of oxygen at the site where the patient suffered the attack, (such as at home, school, work place, or at a common gathering place, such as a sporting venue, shopping mall, bowling alley, or restaurant would significantly reduce the elapsed time between the onset of the patient's attack, and the introduction of oxygen to the patient.

Unfortunately, many facilities that would be well-served by having a supply of oxygen, such as schools, work places, homes, stadiums, restaurants, and other gathering places, do not have such oxygen delivery systems because, among other reasons, no personnel at these facilities are trained sufficiently to be able to properly use the prior art oxygen delivery system. In this regard, since an oxygen delivery system is normally used during an emergency or semi-emergency situation, the situational stress and time-critical nature of the attack rarely gives untrained personnel sufficient time to “read the directions” before beginning use of the device, and further results in a high level of stress to those using the device to give oxygen to a patient. In short, the prior art device is believed by the Applicant to be generally too complicated to be used effectively in an emergency situation by most or all of those who are not either highly trained or frequent users of oxygen delivery devices.

Therefore, a simplified, yet similarly effective oxygen delivery system has the potential to benefit patients suffering such acute attacks because a more simplified device is more likely to be purchased for use at facilities such as homes, schools and the like, and is more likely to be used correctly by the personnel who frequent or operate such home, institutional and commercial establishments.

It is therefore an object of the present invention to provide a regulator/flowmeter incorporating a flow meter, and with provision to allow the delivery of a sigh volume of oxygen for a short period of time. This is commonly referred to as Oxygen Flush.

### SUMMARY OF THE INVENTION

In accordance with the present invention there is envisaged a gas supply regulator/flowmeter for supplying gases to a patient from a gas cylinder, said regulator/flowmeter incorporating means to regulate gas pressure and means to control a main rate of flow of gas, wherein a valve means is provided whereby to provide, upon activation, and downstream of the means to control the main rate of flow of gas, a temporary burst of increased flow of gas to the patient via associated resuscitation or oxygen therapy equipment.

In one preferred embodiment of the invention the regulator/flowmeter provides a single constant volume of main gas flow, for example 8 litres/minute, and the valve means provides the temporary burst of increased gas flow when required.

In another preferred embodiment of the invention a main valve means is provided and is adjustable to provide a plurality of different volumes of main gas flow, as with known regulator/flowmeters, and the valve means is a supplementary valve means to provide the temporary burst of increased gas flow when required.

For patients requiring oxygen a gas flow volume of 8 litres/minute is normally sufficient, whilst the same is normally sufficient for operating a nebuliser for asthma sufferers, as well as for assisting the breathing of sufferers from respiratory problems, whilst the supplementary valve mean is operated when required to give a burst of increased gas flow or open flush. One other advantage of a regulator/flowmeter, which may provide for a single constant volume of gas flow, with the increased gas flow only when required, is that the operator has only two functions to perform in what maybe stressful situations, and that is to turn on the main supply of gas and actuation of the supplementary valve means when a burst of increased gas flow is required. However a main valve means which provides for a plurality of selected constant gas flows as with regard to the other preferred embodiment may be advantageous in some situations.

### BRIEF DESCRIPTION OF THE DRAWINGS

Two preferred embodiments of the invention, will now be described with reference to the accompanying drawings, in which:

FIG. 1 is a schematic longitudinal cross-sectional view through the regulator/flowmeter of one preferred embodiment of the invention, and which, in this preferred embodiment, is cylindrical or “Barrel” shaped.

FIG. 2 is a partly sectioned longitudinal view of a cartridge assembly which regulates the oxygen pressure in the regulator/flowmeter of the first embodiment,

FIG. 3 is a cross-sectional view of the regulator/flowmeter of a second preferred embodiment of the present invention, and

FIG. 4 is a cross-sectional view of a restricter for use in the embodiment of FIG. 3.

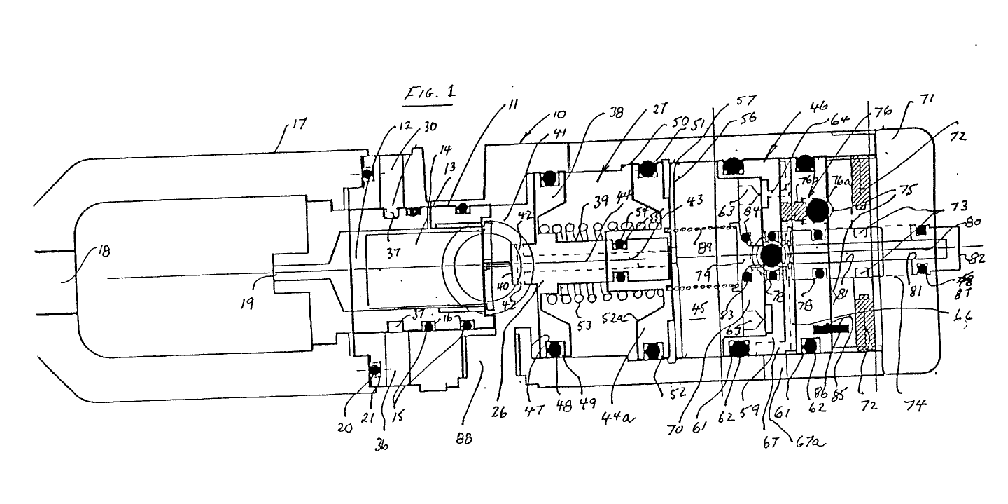

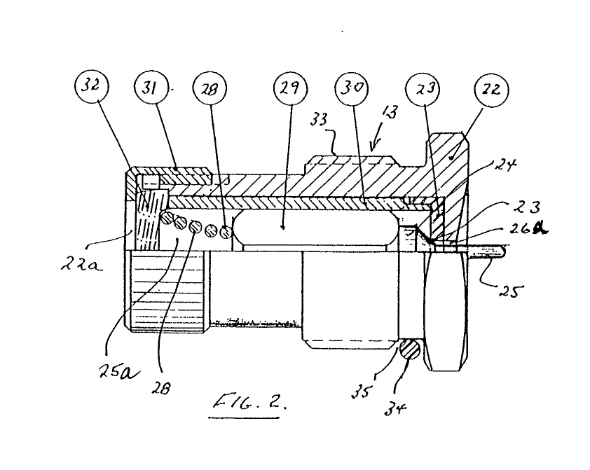

Referring to FIG. 1 of the drawings, the regulator/flowmeter of this preferred embodiment of the invention has a cylindrical housing or body, generally indicated as 10, and known as a barrel regulator/flow meter, although the housing may be of suitable shape other than cylindrical. A cartridge valve holder 11 is inserted at the upstream end of the housing 10, and has a chamber 12, within which a valve assembly 13, in the form of a cartridge, is received, and from which chamber a gauge port 14 is provided for connection to a pressure gauge (not shown) which measures the oxygen pressure within the chamber 12 which is the source pressure (i.e. the gas pressure within an associated oxygen cylinder or bottle). Sealing “O” rings 15 are received within grooves 16 on either side of the gauge port 14 to prevent escape of oxygen between the outside of the cartridge valve holder 11 and the interior of the housing 10. The upstream end of the housing 10 is coupled to an adaptor 17 into which, as is known, a stirrup screw (not shown) is screwed through a threaded aperture 18 to compress cylinder valve against a bodok seal (not shown) into the face of the cartridge valve holder 11 to form a seal to allow oxygen, from the associated oxygen cylinder (not shown), to be admitted to the chamber 12. A sealing “O” ring 20 retained with a groove 21 in the end of the housing 10 and between the housing and adaptor 17 prevents the leakage between the housing and the adaptor. The cartridge valve assembly 13, as shown in FIG. 2, comprises a housing or body 22 with a valve seat 23 against which a valve member 24 seats and which has a stem 25 which is contacted by a piston member 26a of a pressure regulation section 27 (see FIG. 1) of the regulator/flowmeter, whereby the cartridge valve assembly can adjust the oxygen pressure within the regulator/flowmeter. As the valve member 24 lifts off the valve seat 23 to a degree, such by adjustment of the pressure regulation section 27, the oxygen within the interior 25a of the cartridge valve assembly exits through an exit port 26a through which the stem 25 of the valve member 24 extends, and oxygen enters the interior of the cartridge valve assembly through an inlet port 22a in a retaining nut 31. The valve member 24 is biased to a position closing the exit port by a compression coil spring 28 acting against a hollow, cylindrical member 29 extending from the valve member 24. The coil spring 28, and which is guided by a guide member 30, forms a seal with the valve seat 23. The retaining nut 31, which is screwed onto the end of the cartridge valve assembly 13, completes the assembly, whilst a sintered filter 32 is sandwiched between the retaining nut 31 and the rest of the cartridge valve assembly which incorporates a sintered filter.

The exterior of the cartridge valve assembly has a screw threaded section 33 formed thereon which cooperates with an internally threaded section of the cartridge valve holder to hold the cartridge valve assembly in the holder, whilst a sealing “O” ring 34 is received within a groove 35 around the cartridge valve assembly to prevent the escape of oxygen from between the cartridge valve assembly 13 and its surrounding holder 11.

In order to retain the cartridge valve holder 11 within the housing 10, grub screws (not shown) are screwed into cooperating threaded apertures 36 through the wall of the housing 10 to engage in apertures or grooves 37 in, or around, the exterior of the holder 11.

Also, with reference to FIG. 1 of the drawings, and in order to allow adjustment of oxygen pressure by the cartridge assembly 13, the piston member 26 of the pressure regulator/flowmeter section 27 is received within the housing 10 and has a circumferentially extending flange 38 around a stem 39, one end of which has a face 40 which engages the end of the stem 25 of the valve member 24 within the cartridge valve assembly 13, and within a chamber 41 within the housing 10. Radially extending ports 42 in the stem 39 communicate with an axially extending passage 43 through the stem 39 to allow the flow of oxygen through the piston member 26 and an axially extending passage 43 through an associated externally threaded adjustment piston 44a into a chamber 45 communicating with a flowmeter section 46 of the regulator/flow meter.

The piston member 26 is received within an externally outwardly stepped portion 47 of the interior of the housing 10 and has a sealing “O” ring 48 within a circumferentially extending groove 49 to provide a seal between the adjustment piston member 44a and the interior of the surrounding housing 10. The adjustment piston 44a is also received within an externally outwardly stepped portion 50 of the interior of the surrounding housing 10 and also has a sealing “O” ring 51 within a corresponding groove 52 in a flange 52a to provide a seal between itself and the interior of the surrounding housing. A compression coil spring 53 in interposed between the piston member 26 and the adjustment piston 44a.

By adjustment of the positions of the piston member 26 and the adjustment piston 44a, via the compression coil spring 53, the piston member sets the oxygen pressure through the cartridge valve assembly, via the stem 25 of the valve member 24 within that assembly. A sealing “0” ring 54 within a groove 55 around the circumference of the stem of the piston member 26 prevents escape of oxygen between itself and the adjustment piston 44a. An externally threaded locking ring 56 cooperates with an internal threaded portion 57 of the housing 10 and locks the adjustment piston 44a in position.

The flow control section of the regulator/flow meter, and generally indicated as 46, comprises a rotatable flow control valve housing 59 which has circumferentially extending grooves 61 formed therein and in which “O” rings 62 are received to prevent flow of oxygen between the valve housing 59 and the interior of the housing 10. A disc-shaped main flow control valve 70 is provided within the housing 59, and has a series of radially outwardly positioned apertures 63 of varying sizes. A selected one of the apertures communicates with a flow control passage 64 in flow control housing 59 which in turn communicates with a groove 66 around the circumference of the control valve housing and thereafter to an outlet passage 67, which in turn communicates with an outlet passage 67a through the housing 10.

In order to rotate the flow control valve 70 to the required flow rate position, the disc-shaped control valve 70 is coupled to a calibrated control knob 71 via a pair of grub screws 72 engaging in recesses 73 in a shaft 74 for the control valve. In order to lock the control valve 70 in a selected flow control position, the control knob has a series of apertures 75 in which engages a spring loaded ball locking mechanism 76 (ball 76a and spring 76b) carried within the adjacent side of the flow control housing 59.

In order to limit the rotation of the control knob 71, and therefore the flow control position of the control valve 70, the control valve housing 59 has an incomplete circulator slot 85 formed in its face whilst the knob has a pin 86 attached to, or formed as part thereof, which engages in the slot 85 with the extreme ends of the slot defining the degree of rotation of the knob and therefore the degree of rotation of the control valve 70 within its control valve housing.

It will be appreciated that with this preferred embodiment of the invention the main flow control valve 70 provides for a plurality of different volumes of gas flow, as with known regulator/flowmeter, for example zero flow through a low setting of 3 litres/minute through intermediate settings of 3, 8 and 15 litres/minute and through to a maximum setting of 25 litres/minute.

The shaft 74 of the control knob 71 has, in this case three grooves 78 spaced apart around the circumference thereof and each of which receives sealing “O” rings which prevents escape of oxygen between flow control valve (and its shaft 74) and the surrounding flow control valve housing 59.

In accordance with this preferred embodiment of the invention, and in accordance with essence of the invention, the regulator/flowmeter incorporates a supplementary valve to control flow, and in particular an increased flow (burst) of oxygen through the regulator/flowmeter upstream of the flow control section of the regulator/flow meter, and in addition to the normal controlled flow of oxygen through the regulator/flowmeter. This supplementary valve for an increased flow or a burst of oxygen (oxygen flush), involves the use of a valve member 79, having a shaft 80 extending through to a series of passages 81 through the control valve 70; the control valve housing 65; and the control knob 71, to a control button 82 (Oxygen Flush button) whereby the valve member can be lifted off a stepped valve seat 83, against the action of a biasing compression coil spring 89, which seat is formed within the side of the flow control valve 70 facing chamber 45 via an intervening sealing “O” ring 84.

The control button 82 also has a groove 87 around its circumference in which a sealing “O” ring may be received to prevent the escape of oxygen through the part of the passage 81 through the control button 82.

Finally, the housing 10 may have an outlet port 88 communicating with the chamber 41 to allow diversion of regulated oxygen pressure to other equipment, such as a gas pressurized device.

In an alternative preferred embodiment, the main grub screws having different sized passages can be made available to suit the particular flow rate requirements of the user.

The housing includes a chamber 116 for an oxygen flush valve 117 having a housing 118 defining at one end a flush valve chamber 119 and an axial passage 120 for the stem 121 of a flush valve member 122 having a valve head 123. The flush valve chamber 119 communicates with the chamber 141 via a passage 124 through the housing 111. A flush valve transfer port 125 is provided through the flush valve housing 118, and has a first axial portion 125a and a second radial portion 125b which in turn communicated with a circumferential groove 126 extending around the interior of the main housing 111 adjacent the flush valve 117 with intervening “O” ring seals 128. The groove 126 communicates with a flush passage 127 which in turn communicates with the exit port 115.

The underside 123a of the head 123 of the valve member 122 seats on a “O” ring seal 129 contained in an cavity 129a surrounding the valve stem 121 and which cavity in turn communicates with the flush valve transfer port 125. The head of the flush valve member 122 is normally biased by a compression coil spring 130 against the “O” ring seal 128 to close the cavity 129 and thus preventing oxygen flow through the flush valve transfer port 125. The spring may be retained in position by a boss 131 on the outer side of the valve head 123 and received within the adjacent end of the coil spring, whilst the other end of the coil spring is received within a circular recess 132 formed in the adjacent part of the main housing 111.

In operation, when pressure regulated oxygen supply is provided to the chamber 141 a constant flow rate, such as 8 litres/minute, and as set by the size of the passage 112 through the grub screw 113, is supplied through to the exit port 115. When increased oxygen flow rate is required, by way of an oxygen flush, the stem 121 of the flush valve member 122 is pushed axially inwardly of the regulator/flowmeter to lift its head 123 off the “O” ring seal and against the biasing effect of the coil spring 130 and thus exposing the cavity 129a to pressure regulated oxygen in the chamber 141 and thus all pressure regulated gas to flow, by way of a oxygen flush, through the transfer port 125, groove 126 and flush passage 127, and on to the exit port 115.

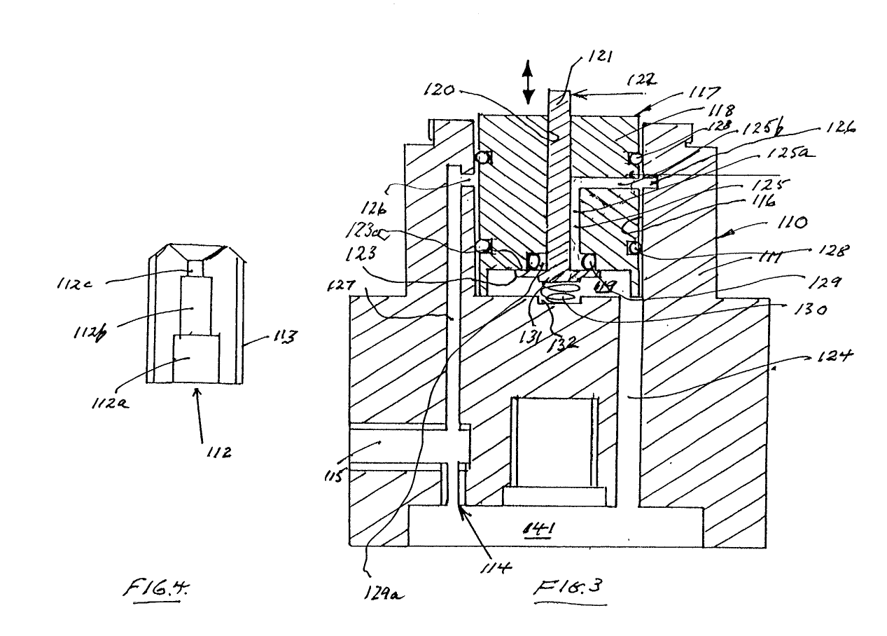

It should be borne in mind that the essence of the invention rests with provision of supplementary valve means to direct an increased flow of gas, such as oxygen, directly to the resuscitation or oxygen therapy equipment and then to the patient, and in addition to a single set flow rate as in the second embodiment of FIGS. 3 and 4 of the accompanying drawings, or one of a plurality of adjustable flow rates as set by the main flow control valve 70 as in the first embodiment described above with reference to FIGS. 1 and 2 of the accompanying drawings.

It should be further borne in mind that it is relatively simple to provide a single set flow rate by simply adding a restricter externally of the outlet from the regulator/flowmeter. However, an oxygen flush in such circumstances is not possible as a restricter provided outside the regulator/flowmeter sets a flow rate which cannot be increased and thus a greater flow rate (oxygen flush) is not possible. With the present invention the oxygen flush is added to the flow beyond the restricter whether it be a single flow rate restricter as is in the embodiment of FIGS. 3 and 4, or an adjustable flow rate restricter as in the embodiment of FIGS. 1 and 2.

1. A gas supply regulator/flowmeter for supplying gases to a patient from a gas cylinder, said regulator/flowmeter incorporating means to regulate gas pressure and mean to control a main rate of flow of gas, wherein a valve means in provided whereby to provide, upon activation, and downstream of the means to control the main rate of flow of gas, a temporary burst of increased flow of gas to the patient via associated resuscitation or oxygen therapy equipment.

2. A gas supply regulator/flowmeter as claimed in claim 1, wherein the means to control the main rate of flow of gas provides a single constant flow rate, and the valve means provides the temporary burst of increased flow of gas.

3. A gas supply regulator/flowmeter as claimed in claim 1, wherein the means to control the main rate of flow of gas is adjustable to provide a plurality of different main flow rates.

4. A gas supply regulator/flowmeter for supplying gases to a patient from a gas cylinder, said regulator/flowmeter incorporating means to regulate gas pressure and means to control a main rate of flow of gas at a single constant flow rate, and wherein a valve means is provided whereby to provide, upon activation, and downstream of the means to control the main rate of flow of gas, a temporary burst of increased flow of gas to the patient via associated resuscitation or oxygen therapy equipment.

5. A gas supply regulator/flowmeter as claimed in claim 4, wherein the main rate of flow of gas through the regulator/flowmeter is controlled by actuation of an outlet valve of an associated gas cylinder.

6. A gas supply regulator/flowmeter as claimed in claim 4, wherein the main rate of flow of gas through the regulator/flowmeter is controlled by main valve means within the regulator/flow meter.

7. A gas supply regulator/flowmeter for supplying gases to a patient from a gas cylinder, said regulator/flowmeter incorporating means to regulate gas pressure and adjustable main valve means to control a main rate of flow of gas at a plurality of different volumes of gas flow, and wherein a supplementary valve means is provided downstream of the means to control the main rate of flow of gas, and to provide, upon activation, a temporary burst of increased flow of gas to the patient via associated resuscitation or oxygen therapy equipment.

8. A gas supply regulator/flowmeter substantially as hereinbefore described with reference to FIGS. 1 and 2 of the accompanying drawings.

9. A gas supply regulator/flowmeter substantially as hereinbefore described with reference to FIGS. 3 and 4 the accompanying drawings.
